(19) United States
(12) Patent Application Publication
(10) Pub. No.: US 2009/0245457 A1
(43) Pub. Date: Oct. 1, 2009

(54) IMAGE GENERATION METHOD AND DEVICE FOR EMISSION COMPUTED TOMOGRAPHY

(76) Inventors: Wataru TAKEUCHI, Hitachi (JP); Keiji KOBASHI, Mito (JP); Isao TAKAHASHI, Hitachi (JP)

Correspondence Address: MATTINGLY & MALUR, P.C., 1800 DIAGONAL ROAD, SUITE 370, ALEXANDRIA, VA, 22314, US

(21) Appl. No.: 12/388,698

(22) Filed: Feb. 19, 2009

(30) Foreign Application Priority Data
Mar. 28, 2008 (JP) 2008-085185

(51) Int. Cl. A61B 6/00; G06K 9/00; H05G 1/60

(52) U.S. Cl. 378/8; 382/131; 378/19

(57) ABSTRACT

A first γ-ray generating in a body, caused by a PET pharmaceutical, and a second γ-ray emitted from a γ-ray source and transmitting through the body are detected with a radiation detector. The emission image information (E image information), E0, E1 and E2, at each of patient motion phases, 0, 1 and 2, which divided a cardiac beat and respiration period, are prepared by using each information obtained from the detected first γ-ray. The transmission image information (T image information), T0, T1 and T2, at each of patient motion phases, 0, 1 and 2, respectively, are prepared, by using each information obtained from the detected second γ-ray. Relative displacements, ([F10], [F20]), are determined by superimposing, on T image information T0, other T image information, T1 and T2. The E image information, E1, E2 are superimposed on the E image information E0, by using this relative displacement.

### BACKGROUND OF THE INVENTION

The present invention relates to an image generation method using an image processing device.

In recent years, representative ones of a radiation image diagnostic device, which non-invasively obtains image information in the body of a subject by applying a radiation measuring technique, include nuclear medicine diagnostic devices, such as a device for positron emission computed tomography (hereinafter, referred to as a PET device) and a device for single photon emission computed tomography (hereinafter, referred to as a SPECT device), and an X-ray CT scanner (hereinafter, referred to as a CT device).

These radiation image diagnostic devices measure a detected physical quantity as an integrated value in the radiation traveling direction, and reconstruct tomographic image information using the physical quantity at each position in the body of a subject, respectively, based on this integrated value. In the X-ray CT scanner, an X-ray source is revolved around a subject and an X-ray emitted from the X-ray source is irradiated from each peripheral position onto a patient who is the subject. In each X ray radiating direction, each X ray passing through the patient is detected by radiation detectors, and the transmissivity of radiation for each radiation direction is calculated based on each X-ray detection signal. By use of the transmissivity of radiation, a spatial distribution of radiation attenuation rates in the body of a patient is topographically imaged. The X-ray CT scanner is widely utilized in the medical field because detailed anatomical images are obtained.

In the nuclear medicine diagnostic devices, such as the PET device and the SPECT device, γ rays caused by the radiopharmaceuticals injected into a patient and emitted from the body of the patient is detected by radiation detectors, and a distribution of the radiopharmaceuticals in the body of the patient is topographically imaged using measurement information which is obtained based on the detected γ ray detection signal. The obtained tomographic image information is used for diagnosis of a metabolic function and a physiological function.

Since these techniques need to process a large amount of data, the quality of the obtained image information is limited due to data processing constraints. However, with a rapid advance in the signal-processing circuits and computer technology in recent years, high quality image information can be obtained.

In the X-ray CT scanner, as described above, the transmissivity of X-ray in various directions is obtained using X-ray detection signals output from radiation detectors. By processing the measurement data of the transmissivity using an image reconstruction algorithm, such as the filtered back projection method described in IEEE Transactions on Nuclear Science, Vol. NS-21, PP. 228 and 229, for example, a distribution of linear attenuation coefficients at each region in the body can be obtained as three-dimensional image information.

In examination (PET examination) using the PET device, the radiopharmaceuticals (hereinafter, referred to as the PET radiopharmaceuticals) labeled with a positron emitter (¹⁵O, ¹³N, ¹¹C, ¹⁸F, or the like), the radiopharmaceuticals specifically accumulating at a particular region in the body, is administered to a patient and then a distribution of the PET radiopharmaceuticals in the body of the patient is examined. A positron emitted from the PET radiopharmaceuticals administered to the patient couples with an electron nearby in the body and emits a pair of annihilation γ rays (hereinafter, referred to as paired γ rays) with an energy of 511 KeV. Since each of the paired γ rays is emitted in directions substantially opposite to each other, both γ rays are measured with the respective radiation detectors and a coincidence measurement is performed using each information which is obtained based on the respective γ ray detection signals output from these detectors. Thus, it is possible to identify on which straight line the positron annihilation event occurred. After detecting a statistically sufficient number of paired γ rays in this manner, a distribution of frequency of occurrence of the paired γ rays, i.e., a distribution of the PET radiopharmaceuticals in the body of a patient, can be topographically imaged using an image reconstruction algorithm, such as the above-described filtered back projection method.

The measurement of γ rays caused by the PET radiopharmaceuticals in the body is referred to as emission measurement (hereinafter, referred to as E measurement), and image information which is reconstructed based on γ rays detection signal obtained by the emission measurement is referred to as emission image information (hereinafter, referred to as E image information). Usually, E image information is simply referred to as a PET image, but in this specification it is referred to as the E image information in order to discriminate from the later-described transmission image information (hereinafter, referred to as T image information). Moreover, a series of processes from E measurement to reconstruction are collectively referred to as emission imaging.

In examination (PET examination) using a PET device, the examination requiring quantitativity, measurement (transmission measurement (hereinafter, referred to as T measurement)) called “transmission” using a γ ray source that is a transmission radiation source provided in the PET device is also performed separately from E measurement. Attenuation of a γ ray in the PET measurement refers to a phenomenon that a γ ray caused by the radiopharmaceuticals is not detected as coincidence measurement data effective in imaging as a result of the γ ray interacting with materials in the body before the γ ray goes outside the body of a patient. A process to correct the amount of attenuation of a γ ray is referred to as attenuation correction, and is implemented in most of the PET examinations at present.

The attenuation correction is usually performed using data obtained in T measurement in which a γ ray source is revolved around a patient lying on a bed. It is also possible to use an X-ray source described in IEEE Transactions on Nuclear Science, Vol. NS-21, PP. 228 and 229, instead of the γ ray source.

The major factors degrading the image quality of E image information include an effect of a patient's movement (patient motion). The patient motions include involuntary respiration, a periodic movement associated with heartbeat, and a voluntary posture change. For example, in PET examination, measuring time usually takes nearly several minutes to several tens of minutes, so it is difficult to suppress a patient motion without stressing the patient. In particular, it is impossible to perform PET examination over a long time while the respiration is stopped, so it is difficult to suppress the movement (respiratory motion) caused by respiration. Therefore, it is known that E image information in a region close to the lung field will blur. In particular, in the cases where a subject to be examined in PET examination is the heart, it is impossible to suppress and control pulsation. Therefore, the image quality of E image information will be damaged by the influence of the motion of the heart.

As a method of correcting the blurring of image information associated with periodic motions, such as respiration and heartbeat, a method called gated-acquisition is known. The gated-acquisition is a method comprising the steps of sorting E measurement data measured over a plurality of motion cycles in each data of each motion phase, and reconstructing E image information for each motion phase using these sorted data, respectively. For example, in the gated-acquisition with respect to a respiratory motion, phase information of respiration is obtained, for example, by capturing a tiny change in breath temperature as described in Journal of Nuclear Medicine, Vol. 45, No. 2, PP. 214-219, or by tracking the chest motion with an infrared stereoscopic camera as described in Journal of Nuclear Medicine, Vol. 43, NO. 7, PP. 876-881, and then the data obtained by E measurement based on this phase information is sorted for each motion phase. In the gated-acquisition of a heart, a trigger signal indicative of the phase of a cardiac beat is usually obtained from ECG (Eco Cardiography). The gated-acquisition is at present commonly performed with respect to the examination of a heart in particular (JP-A-2004-65982 and JP-A-2000-107174), and also has begun to be attempted also with respect to respiratory motion.

According to this gated-acquisition method, it is theoretically possible to compensate the effects associated with periodic motions. However, the required examination time is long. Since the long time examination is a pain to a patient (in most cases, a patient having a disease), the measurement is usually finished in around 20 to 30 minutes. In this case, as Journal of Nuclear Medicine, Vol. 45, No. 2, PP. 214-219 points out, it is difficult to obtain data required for generating E image information having a sufficient statistical accuracy because the measurement is performed for several minutes at most with respect to each motion phase. Therefore, the obtained E image information includes a lot of statistical noises. Although the statistical accuracy of E image information can be improved by reducing the number of motion phases per motion cycle, the patient motion in one motion phase will increase and consequently a significant improvement of the accuracy as the motion compensation cannot be expected. Furthermore, in order to compensate both of the cardiac beat and the respiration, it is necessary to perform measurement for each motion phase and thus the PET examination time will increase further.

In particular, in PET examination, attenuation correction is often performed and thus ideally E measurement and T measurement should be performed in an identical motion phase, respectively. However, as described above, a long time measurement is required in order to perform gated-acquisition. Accordingly, T measurement is usually performed while stopping breathing or while breathing, and then by use of this single data, the attenuation correction of an emission image of gated-acquisition with a plurality of phases is performed. So, the attenuation correction will be performed using the measurement data obtained under different conditions, and artifacts causes by a positional deviation of the patient between E measurement and T measurement will occur in the obtained image information.

As other methods of compensating the respiratory motion, a method using a combined PET/CT device (see Journal of Nuclear Medicine, Vol. 45, No. 8, PP. 1287-292) is available. In Journal of Nuclear Medicine, Vol. 45, No. 8, PP. 1287-292, in the combined PET/CT device, X-ray CT imaging is performed in a cine mode to obtain E image information for each respiration phase, and then in generating the respective phase image information of the E image information of gated-acquisition, the attenuation correction is made using an X-ray CT image of the corresponding respiration phase. Although gated-acquisition of “transmission” is usually not performed, IEEE Transactions on Nuclear Science, Vol. 44, No. 6, PP. 2473-2476 reports on this as far as inventor knows at the present. The gated-acquisition of IEEE Transactions on Nuclear Science, Vol. 44, No. 6, PP. 2473-2476 takes a long time of 20 minutes. The gated-acquisition of the heart using a CT device is described in JP-A-2004-65982 and JP-A-2000-107174. The gated-acquisition of the heart is the gated-acquisition of a single periodic motion. In order to compensate cardiac beat, because of the magnitude of this motion, it is also necessary to compensate the respiratory motion as described above, and it is easily imagined that if the gated-acquisition is simply performed on these two motions, a longer measuring time will be required. Note that, IEEE Transactions on Medical Imaging, Vol. 18, P. 657 describes Fourier re-binning method and a three-dimensional successive approximation reconstructing method which are methods for reconstructing tomographic image information. IEEE Transactions on Medical Imaging, Vol. 18, PP. 712-720 discloses a nonlinear superimposing technique (non-rigid image registration method) of two pieces of image information.

### SUMMARY OF THE INVENTION

By performing gated-acquisition during E measurement as described above, it is theoretically possible to compensate effects associated with periodic motions. However, in order to obtain data with sufficient statistical accuracy for each motion phase, the required measuring time will increase. However, a long time PET examination is a pain to a patient (in most cases, a patient having a disease).

With regard to problems (1) blurring of E image information and (2) a decrease in the quantitativity of E image information, Journal of Nuclear Medicine, Vol. 45, No. 2, PP. 214-219 describes that it is possible to improve statistical accuracy by nonlinearly transforming E image information obtained by gated-acquisition into the image information of a definite phase and superimposing the resultant pieces of information onto each other and superimposing the pixel values. However, Journal of Nuclear Medicine, Vol. 45, No. 2, PP. 214-219 does not recognize the difficulty in transforming E image information and superimposing these pieces of information onto each other. Unlike the anatomical images, such as image information reconstructed by X-ray CT and T image information, E image information is not intended to depict a structure in the body. For this reason, usually, it is extremely difficult to transform E image information for each motion phase and superimpose these pieces of information onto each other.

It is an object of the present invention to provide an image generation method which can obtain clear functional image information of a living organ, targeting at a region that moves under the influence of respiration and cardiac beat, in a short time.

According to an aspect of the present invention achieving the above-described objective, the image generation method comprises the steps of: generating first tomographic image information in a plurality of time intervals, which are defined by individually combining a plurality of first phases obtained by temporally dividing a certain cycle with a plurality of second phases obtained by temporally dividing other cycle different from the certain cycle; generating second image information of a structure of a living organ imaged in the plurality of time intervals; superimposing the second image information of other time interval onto the second image information of the one certain time interval among the plurality of time intervals, and calculating relative displacement information between these pieces of second image information; and generating first superimposed image information by superimposing the first image information of the other time interval onto the first image information of the one certain time interval by use of the relative displacement information.

According to the present invention, it is possible to obtain clear functional image information of a living organ in a short time, targeting at a region that moves under the influence of respiration and cardiac beat.

### BRIEF DESCRIPTION OF THE DRAWINGS

FIG. 1 is an explanatory view of a respiratory motion compensation method.

FIGS. 2A and 2B show a revolution of a γ ray source and a motion cycle, where FIG. 2A is an explanatory view showing a revolving state of the γ ray source, and FIG. 2B is an explanatory views showing the motion cycle.

FIG. 3 is a schematic configuration diagram of a device for positron emission computed tomography as an example.

FIG. 4 is a detailed configuration diagram of the device for positron emission computed tomography shown in FIG. 3.

FIG. 5 is a detailed configuration diagram of a tomographic image generation device and a motion compensation device shown in FIG. 4.

FIG. 6 is a vertical cross-sectional view of the imaging device shown in FIG. 3.

FIG. 7 is an explanatory view when a detector unit is mounted in the imaging device.

FIG. 8 is a perspective view showing a detailed configuration of a detector unit shown in FIG. 4.

FIG. 9 is a view showing a detailed configuration of a module board shown in FIG. 8.

FIG. 10 is an explanatory view showing a two-dimensional table in a storage device shown in FIG. 4.

### DETAILED DESCRIPTION OF THE INVENTION

Hereinafter, a device for positron emission computed tomography which is an embodiment of the present invention will be described suitably referring to the accompanying drawings.

### Embodiment 1

Blurring of image information due to a patient motion has been a problem in the following points.

(1) The body contour and the contour of an internal organ and a tumor will blur.

(2) In a region having a high degree of accumulation of radiopharmaceuticals, the degree of accumulation is underestimated than the actual one and the diagnostic performance will degrade.

(3) superimpose of image information with a plurality of modalities does not work well.

The above problems are fundamental influences which a patient motion has on the image information. At present when the spatial resolution of a PET device has dramatically increased, (1) and (2) are of interest as the major factors damaging the image quality. With regard to (3), even at present when superimpose of image information has become popular due to the recently-emerged combined PET/CT device (see Journal of Nuclear Medicine, Vol. 45, No. 8, PP. 1287-292), mainly in the fields of radiation therapy, biopsy, and the like which need to identify the position of a malignant tumor more accurately, there is a strong demand to solve this problem.

The problem of (1) makes difficult the radiation treatment planning and the evaluation of effects of a medical treatment because an internal organ and a malignant tumor or other illness region are blurred. Moreover, a malignant tumor which is small and has a relatively low degree of accumulation also appears clear in the displayed E image information if there is no patient motion. However, if there is a patient motion, the relevant malignant tumor is hidden in statistical noise contained in E image information and even recognition of its presence is difficult.

The problem of (2) is that the degree of accumulation is usually underestimated in a malignant tumor having a large motion, for example. In E image information, since the degree of accumulation shown in a certain pixel at a position where a patient motion is large is a time-averaged degree of accumulation of peripheral regions, the degree of accumulation will consequently deviate from the actual value. This lowers the degree of accumulation as described above.

The problem of (3) arises when E image information obtained by imaging for several minutes while naturally breathing and an X-ray CT image taken in a short time while stopping breathing are superimposed onto each other. Although useful in diagnosis, this superimposition image information appears as if there is a deviation on the order of 1 cm at maximum on the image around the lung field. This is because the way to deal with respiration differs between the PET examination and the ordinary X-ray CT examination and accordingly positional correspondence therebetween cannot necessarily be established. This problem is pointed out also in the PET/CT device and there is a need to solve this problem.

As described above in the E measurement, by performing gated-acquisition, it is theoretically possible to compensate effects associated with periodic motions. However, in order to obtain data with sufficient statistical accuracy for each motion phase, the required measuring time will increase. However, a long time PET examination is a pain to a patient (in most cases, a patient having a disease).

Journal of Nuclear Medicine, Vol. 45, No. 2, PP. 214-219 describes that, with regard to the problems of (1) and (2), it is possible to improve statistical accuracy by nonlinearly transforming E image information obtained by gated-acquisition into image information of a definite phase and superimposing these pieces of information onto each other and superimposing their pixel values. However, Journal of Nuclear Medicine, Vol. 45, No. 2, PP. 214-219 doesn't discuss the specific method. Unlike anatomical images, such as image information reconstructed by X-ray CT and T image information, E image information is not intended to depict a structure in a body. For this reason, usually, it is extremely difficult to transform E image information for each motion phase and superimpose these pieces of information onto each other.

Furthermore, for example, as Journal of Nuclear Medicine, Vol. 45, No. 2, PP. 214-219 points out, also when gated-acquisition is performed in E measurement, in the PET device the attenuation correction is performed using T measurement data obtained by non-gated-acquisition and in the combined PET/CT device the attenuation correction is performed using an X-ray CT image taken while stopping breathing or while breathing. For this reason, due to a difference in the respiratory conditions of a patient between the data obtained by E measurement and the data obtained by T measurement, such a problem will occur that artifacts occur in the attenuation compensation or that the quantitativity degrades because the compensation is not performed correctly. Journal of Nuclear Medicine, Vol. 45, No. 2, PP. 214-219 suggests that T measurement using a γ ray source or the gated-acquisition in X-ray CT is a solution to this problem. However, Journal of Nuclear Medicine, Vol. 45, No. 2, PP. 214-219 has not shown a specific method of this solution.

In the method using a combined PET/CT device described in Journal of Nuclear Medicine, Vol. 45, No. 8, PP. 1287-292, X-ray CT imaging originally requiring high dose of irradiation is performed for a relatively long time and thus a problem of high radiation exposure will arise. This method cannot fundamentally solve the problems of (1) and (2) either because a problem of insufficient statistical accuracy in E image information described in Journal of Nuclear Medicine, Vol. 45, No. 2, PP. 214-219 remains. Furthermore, in this combined PET/CT device, the E measurement by PET and the measurement by X-ray CT temporally deviate from each other. For this reason, the combined PET/CT device may receive an influence based on temporal deviations of a posture change, a fluctuation of respiration, and the like of a patient in the both measurements, and may generate deviations in the attenuation correction and the alignment of image information.

A method of accurately implementing PET examination in a short time that takes into consideration the cardiac beat in addition to the respiratory motion and compensates both the cardiac beat and the respiratory motion is not known. In order to compensate cardiac beat, because of the magnitude of this motion, it can be easily imagined that if gated-acquisition is simply performed on two motions of cardiac beat and respiratory motion, a longer PET examination time will be required. Time required for gated-acquisition is desired to be reduced.

It is an object of the present invention to provide a device for positron emission computed tomography which can obtain clear emission image information in a short time, targeting at a region that moves under the influence of respiration and cardiac beat, and a tomographic image generation method of the same.

As described above, in PET examination around the lung field, the removal of the influence of patient motions, particularly respiratory motion, is indispensable in order to obtain high quality E image information. For this purpose, it is preferable to solve also the problems shown below other than the above-described ones of (1)-(3).

(4) Do not force a subject to unreasonably stop breathing.

(5) Do not cause an artifact or quantitative degradation due to attenuation correction.

(6) Perform examination in such a short time that will not give a pain to a subject.

As described in Journal of Nuclear Medicine, Vol. 45, No. 2, PP. 214-219, it is suggested that image information with high statistical accuracy and with compensated respiratory motion could be obtained by a method comprising the steps of: gated-imaging E image information; superimposing image information of the respective motion phases onto image information of one certain motion phase while nonlinearly distorting and transforming the same; and adding up pixel values of the corresponding pixels. If this can be achieved, (1)-(4) and (6) among the above-described problems can be solved. However, as described above, it is usually impossible to superimpose nonlinear image information based on E image information having few pieces of anatomical information. Therefore, a certain idea is required to solve this problem. Moreover, preferably, the problem of (5) also needs to be solved.

The inventors of the present invention studied a technique capable of performing nonlinear superimpose of E image information. The results of this study will be described hereinafter.

In order to obtain measurement data with high statistical accuracy for each motion phase of periodic motions (a respiratory motion and a beat of the heart) in a subject, a PET examination over a long time is required. For this reason, it is difficult to obtain this measurement data within a measuring time which an ordinal subject can withstand without feeling a pain. Then, the present inventors focused attention on a fact that statistical accuracy can be improved by superimposing E image information for each motion phase obtained by gated-acquisition (hereinafter, referred to as E gated-acquisition) in emission measurement of a shorter time onto the image information of one certain motion phase to superimpose the respective pixel values. However, E image information originally has few pieces of anatomical information, and E image information for each motion phase, which can be obtained based on data obtained by gated-acquisition of a short time, lacks of statistical accuracy. For this reason, it is difficult to perform nonlinear superimpose of E image information directly between E image information for each motion phase.

The reason why PET examination which performs gated-acquisition in transmission measurement (hereinafter, referred to as T gated-acquisition) takes a long time is that a γ ray source is revolving around a subject. In order to reconstruct E image information having fewer artifacts, it is necessary to emit a γ ray onto a subject in a substantially uniform time from every angular orientation.

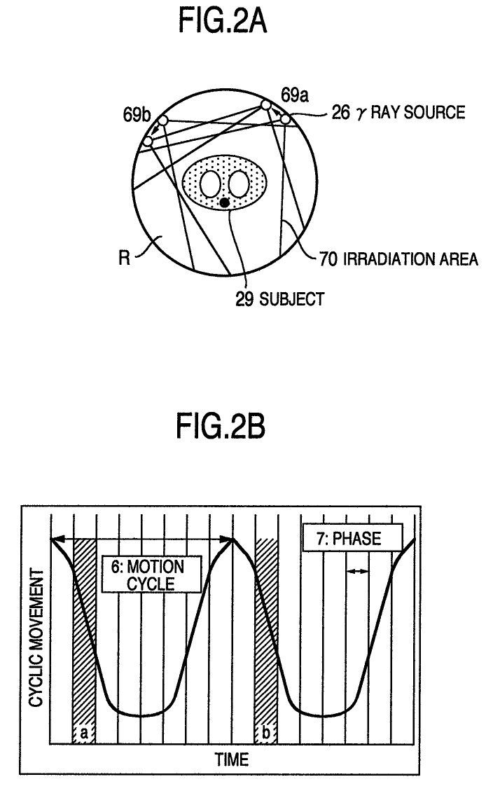

FIGS. 2A and 2B show the revolution of a γ radiation source and a motion cycle, where FIG. 2A is an explanatory view illustrating the revolving states of a γ ray source and FIG. 2B is an explanatory views illustrating the motion cycle. Although it is necessary to emit a γ ray onto a subject in a substantially uniform time from every angular orientation, a radiation source 26 which is the γ ray source is revolving around a patient 29 independently of the motion phases a, b which are subject to gated-acquisition. Therefore, when focusing on one certain motion phase, the circumferential position of the radiation source 26 are discontinuously varying. That is, the positions 69a and 69b of the radiation source 26 in the motion phases a and b shown in FIG. 2B which are an identical motion phase are not guaranteed to take all the positions of the γ ray source in its continuity and in imaging during a predetermined period. That is, for example, when the rotational cycle of an external radiation source has been synchronized with the motion cycle, imaging can be performed only from a certain definite position of the external radiation source no matter how much time is spent. Thus, it takes a considerable long time to obtain the data of irradiation for a substantially uniform time from various angular orientations in all the motion phases.

After performing various studies, the present inventors noticed that T gated-acquisition may be performed in parallel with E gated-acquisition so as to reconstruct T image information for each motion phase. As a result, the present inventors have found a new method of indirectly obtaining emission superimposed image information (hereinafter, referred to as E superimposition image information) by nonlinearly superimposing E image information for each motion phase onto each other by use of transmission superimposition image information (hereinafter, referred to as T superimposed image information) obtained by nonlinearly superimposing T image information for the each motion phase onto each other. With this new method, nonlinear superimpose of E image information can be performed.

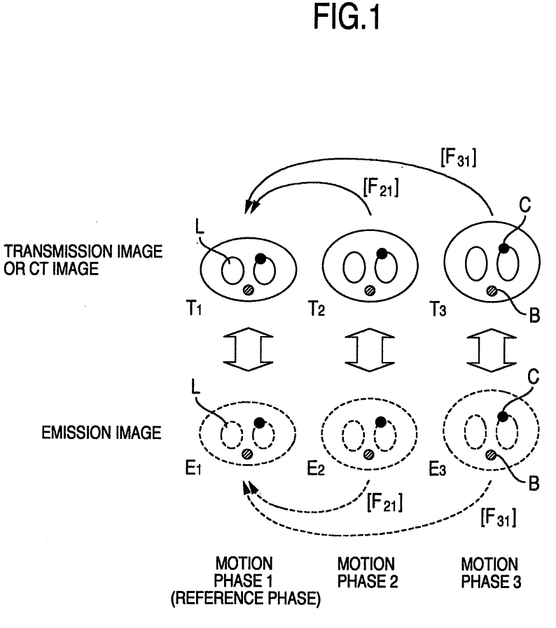

Now, the basic concept of the above-described new method which the present inventors have found is described in detail using an explanatory view of a respiratory motion compensation method of FIG. 1. For ease of understanding, FIG. 1 shows a case where a respiration cycle which is a motion cycle is divided into three motion phases, as an example. In FIG. 1, B represents the backbone, C represents a malignant tumor, and L represents the lung.

T₁, T₂, and T₃ in the upper side of FIG. 1 are the pieces of T image information corresponding to motion phases 1, 2, and 3, respectively. These pieces of image information are obtained by reconstructing second packet information obtained by T gated-acquisition. Here, the pieces of information obtained in T gated-acquisition are second detection information including the later-described time information and radiation source positional information, and motion phase information to which the time information is provided. E₁, E₂, and E₃ in the lower side of FIG. 1 are the pieces of E image information corresponding to the motion phases 1, 2, and 3, respectively. These pieces of image information are obtained by reconstructing first packet information obtained by gated-acquisition. Here, the pieces of information obtained in E gated-acquisition are first detection information including the later-described time information, and motion phase information to which the time information is provided. Since the T image information T₁, T₂, and T₃ are anatomical image information, the contour of a bone, an internal organ, or the like is clear, and on the other hand since the E image information E₁, E₂, and E₃ are functional image information, the contour is not clear. In FIG. 1, the contour of T image information is indicated with a solid line, and the contour of E image information is indicated with a dashed line.

As the γ ray source, a radiation source emitting a γ ray of an energy different from the energy of a γ ray that is generated in the body of a subject due to positron annihilation is used, and a radiation detector, such as a semiconductor radiation detector, with an excellent energy resolution is used. Since a radiation detection signal output from the radiation detector is discriminated based on the energy, E measurement and T measurement can be performed in parallel. The respective data (the later-described first packet information and second packet information) obtained and discriminated by these measurements are sorted for each motion phase based on the acquired motion phase information as in the known gated-acquisition. By use of each sorted data, E image information and T image information for each motion phase are reconstructed, respectively. Thus, the T image information T₁, T₂ and T₃ and the E image information E₁, E₂, and E₃ can be obtained.

Although depending on the number of divided motion phases, T gated-acquisition will usually take a very long time. However, by intentionally shifting a relationship between the cycle of a motion phase and the rotational cycle of the γ ray source, a short-time gated-acquisition is possible also in T measurement. In the simulation of the present inventors, when the number of motion phases per respiration cycle is set to 8, if the rotational cycle of the γ ray source is set to (integer ±0.1) times the respiration cycle of a subject, T measurement data that satisfies the reconstruction conditions in around 10 minutes at most can be obtained in the case where the bed is positioned at one certain position.

The respective E image information and T image information obtained for each motion phase by E gated-acquisition and T gated-acquisition, in which a relationship between the cycle of a motion phase and the rotational cycle of a γ ray source is intentionally shifted, are the image information obtained when these measurements are performed in parallel (simultaneously) onto an identical region (e.g., a region influenced by a patient motion) of the same subject. There is substantially no difference in the conformation of the subject between E measurement and T measurement performed in parallel. That is, a spatially relative displacement between two pieces of E image information corresponding to two motion phases in E gated-acquisition is the same as that between two pieces of T image information corresponding to these two motion phases in T gated-acquisition. For example, a spatially relative displacement F₂₁ between E image information E₁ of a motion phase 1 (reference phase) and E image information E₂ of a motion phase 2 in E gated-acquisition is the same as the displacement between T image information T₁ of the reference phase and T image information T₂ of the motion phase 2. For this reason, by calculating information (e.g., F₂₁ or F₃₁) on this relative displacement between two motion phases in T gated-acquisition and then applying this information of the relative displacement to the two pieces of E image information in two motion phase spaces, it is possible to nonlinearly superimpose E image information of one of the motion phases onto E image information of other motion phase (e.g., reference phase space). As a result, nonlinearly-superimposed emission superimposed image information can be obtained.

Note that, T image information can acquire a body contour, anatomical information of a lung field, a bone, and the like, so by using the nonlinear superimpose technique described in IEEE Transactions on Medical Imaging, Vol. 18, PP. 712-720, for example, T image information of the respective motion phases can be superimposed onto an image of the reference phase while being nonlinearly distorted.

FIG. 1 shows a process of obtaining emission superimposed image information. In FIG. 1, a respiration phase corresponding to a state where a subject exhales is set to the reference phase, and T image information in this phase is set to the reference image information T₁. When T image information Tⱼ of other respiration phase (denoted as phase j) is spatially superimposed onto the reference image information T₁, the T image information Tⱼ superimposed onto the reference image information T₁ can be expressed as a mapping function as in Equation (1) (for ease of description, the number of divided phases is set to 3).

Tⱼ₁=Fⱼ₁·Tⱼ (j=2, 3, . . . , n) (1)

Here, Fⱼ₁ is a transformation matrix representing a relative displacement from the image Tⱼ corresponding to the motion phase j to the reference phase image T₁. The T superimposed image information Tⱼ₁ is T image information obtained by mapping T pixel information Tⱼ of the motion phase j onto the conformation of the T image information T₁ of the motion phase 1. These pieces of T image information are expressed in vector, respectively. Superimpose of E image information having unclear conformation is performed using the transformation-matrix Fⱼ₁ obtained from the superimpose of T image information. Superimpose of E image information of other motion phase onto the reference image information E₁ performed using this transformation matrix is expressed by Equation (2).

Eⱼ₁=Fⱼ₁·Eⱼ (j=2, 3, . . . , n) (2)

Here, the E superimposed image information Eⱼ₁ is E image information obtained by nonlinearly superimposing the E image information Eⱼ of the motion phase j onto the reference image information E₁ of the motion phase 1. E image information E₁′ with high statistical accuracy can be obtained by adding up each pixel value corresponding to the thus obtained E superimposed image information as in Equation (3).

$${E}_{1}^{′} = \frac{{E}_{1} + {\sum }_{j = 2}^{n} {E}_{j 1}}{n} \qquad ( 3 )$$

Since E measurement and T measurement are performed in parallel and the respective time instances of a certain motion phase in T measurement and a motion phase in E measurement corresponding to this motion phase are substantially simultaneous, accurate attenuation correction with few positional deviations of a subject can be performed also in the attenuation correction that is typically used in PET examination.

With the above processing, PET image information with higher statistical accuracy can be obtained.

As described above, based on a relative displacement between corresponding pieces of T image information in two motion phases, the corresponding pieces of E image information in these motion phases are nonlinearly superimposed onto each other, and thereby problems other than the above-described (5) can be improved. Since T image information having the conformation coinciding with that of E image information can be obtained for each motion phase, the problem (5), i.e., the problem caused by deviations of the conformation at the time of attenuation correction, can be solved by the projection calculation based on these pieces of T image information or by performing attenuation correction using measurement data (projection data) which was used in reconstructing T image information.

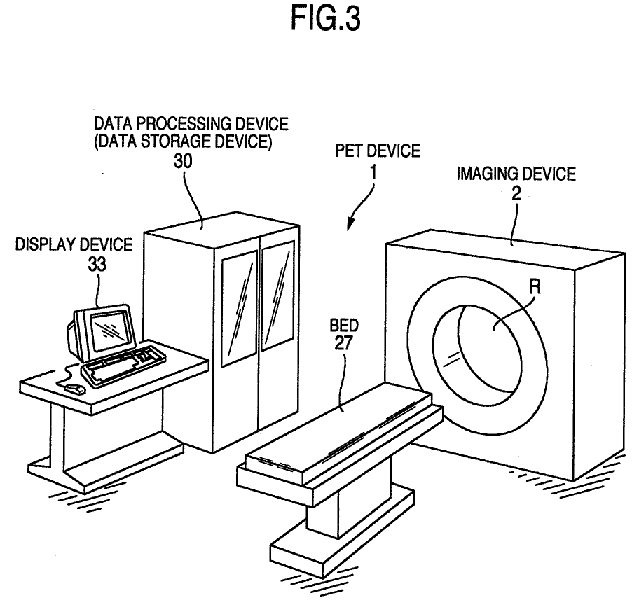

In the respiratory motion compensation method (motion compensation method) described above, the later-described data processing device 30 of FIG. 3 performs the calculation processing. On the basis of a relative displacement between T image information in certain two motion phases, E image information corresponding to these two motion phases are superimposed onto each other, and the resultant image information is output to a display device 33 or is output to a storage device 35 for a process of further superimposing another display information thereon and displaying this, and the resultant information is recorded.

### Embodiment 2

A device for positron emission computed tomography 1 (PET device) which is a preferable embodiment of the present invention will be described using FIG. 3 to FIG. 10. FIG. 3 is a schematic configuration diagram of the device for positron emission computed tomography of the embodiment. As shown in FIG. 3, the PET device of this embodiment comprises an imaging device 2, a bed 27 for supporting the patient 29 who is a subject, the data processing device 30, and the display device 33.

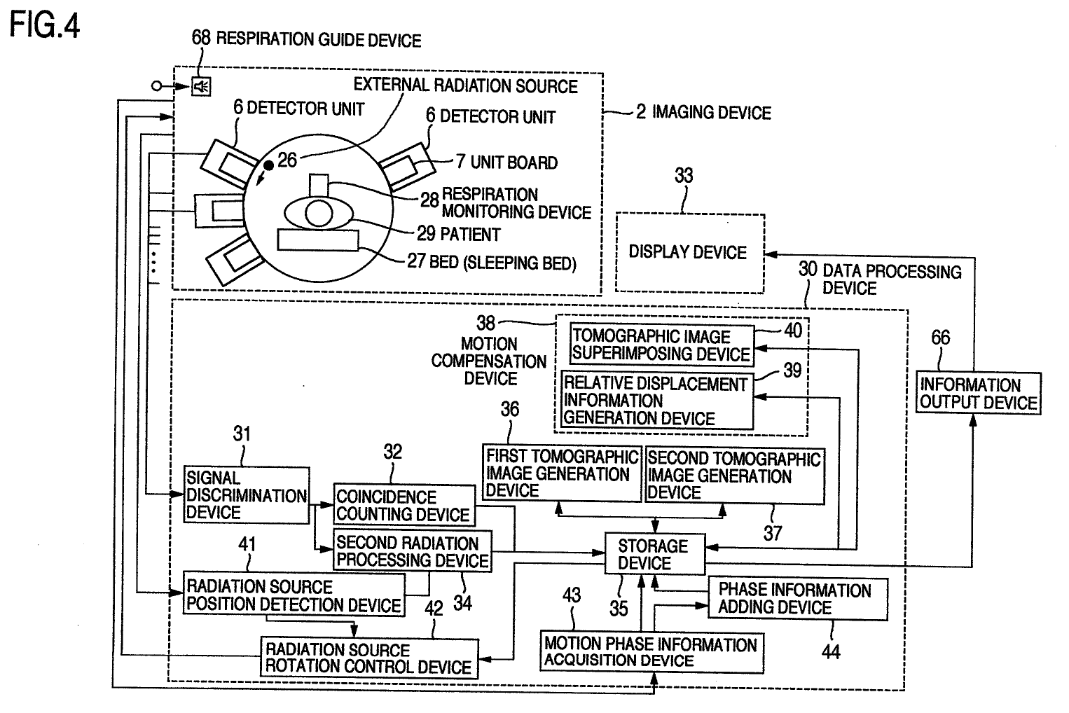

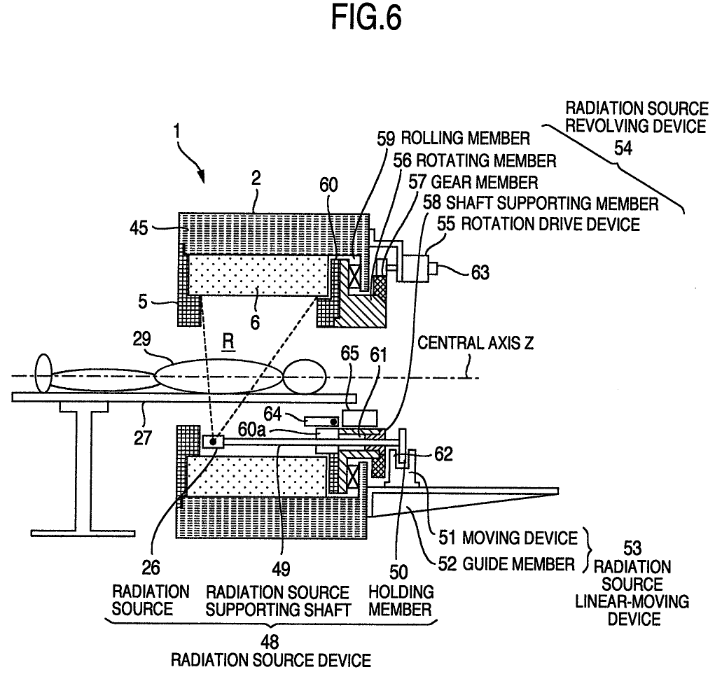

The imaging device 2 includes a housing 45 (see FIG. 6) surrounding a measurement space R, and a plurality of detector units 6 (see FIG. 4 and FIG. 6) arranged so as to surround the measurement space R.

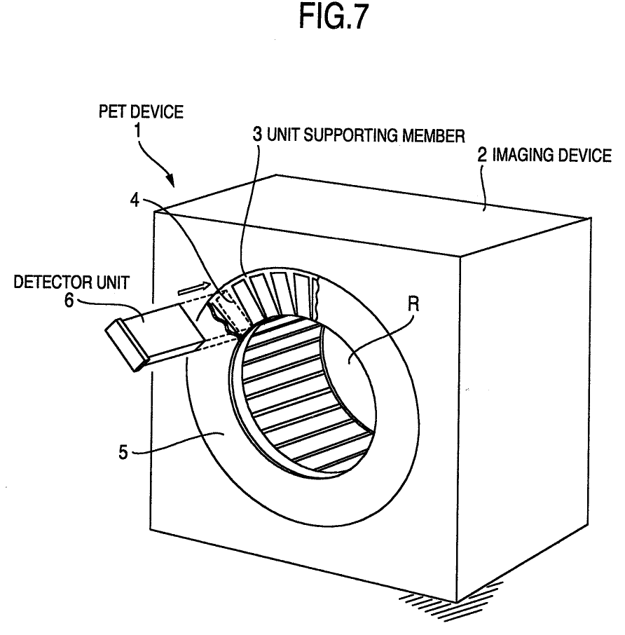

FIG. 7 is an explanatory view when the detector unit is attached to the imaging device. The detector unit 6 is arranged surrounding the measurement space R and held by a unit supporting member 3 installed in the housing 45. These detector units 6 are inserted in a plurality of openings 4, which are provided in the unit supporting member 3 and circumferentially arranged. After the detector units 6 are mounted in the opening 4, an annular front end shield 5 is attached to the housing 45 so as to cover the front of the detector unit 6.

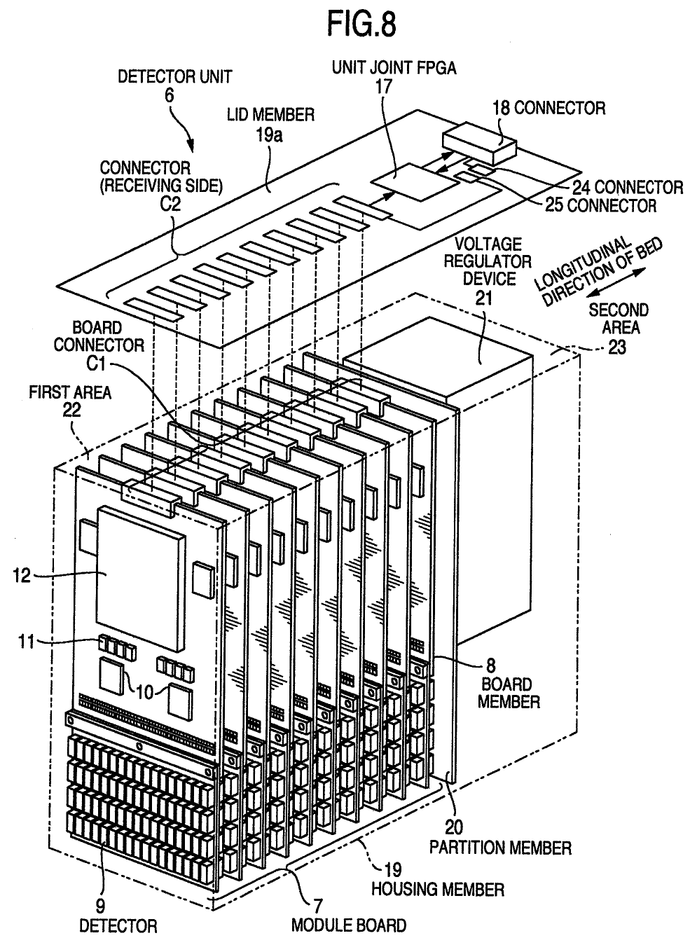

FIG. 8 is a perspective view showing a detailed configuration of the detector unit 6. The detector unit 6 includes a housing member 19 having an internal space formed therein and forming a rectangular parallelepiped, a plurality of semiconductor radiation detectors (hereinafter, referred to as detectors 9) arranged in the housing member 19, a plurality of analog ASICs 10 and digital ASICs 12 serving as a signal processing device, and a voltage regulator device 21. ASIC is an Application Specific IC. A plurality of detectors 9, a plurality of analog ASICs 10, a plurality of digital ASICs 12, an analog digital converter (ADC 11) which converts an analog signal to a digital signal are mounted on a board member 8. One module board 7 includes a plurality of detectors 9, a plurality of analog ASICs 10 and digital ASICs 12, and the board member 8. The space in the housing member 19 is divided into a first area 22 and a second area 23 by a partition member 20 arranged therein. A plurality of module boards 7 are arranged in the first area 22, and are removably attached to the housing member 19. These module boards 7 are arranged so that the respective board surfaces (where the detector 9, the digital ASIC 12, and the like are installed) face to the longitudinal direction of the housing member 19, i.e., the longitudinal direction of the bed 27 (the body axis direction of the patient), and are arranged in parallel in this longitudinal direction. The voltage regulator device 21 is arranged in the second area 23, and is attached to the housing member 19.

Each detector 9 uses cadmium tellurium (CdTe) in a semiconductor device portion excellent in energy resolution. In the semiconductor device portion, gallium arsenide (GaAs), lead iodide (PbI₂), thallium bromide (TIBr), cadmium zinc telluride (CZT), or the like may be used. The semiconductor device portion may use a combination of the above-described materials. Each detector 9 detects a γ ray (a first γ ray) of 511 keV caused by the PET radiopharmaceuticals and emitted from the patient 29, and a γ ray (a second γ ray) emitted from a radiation source 26 (see FIG. 4 and FIG. 6), which is the later-described radiation source, and passing through the patient 29. These detectors 9 are positioned nearer to the measurement space R side than the analog ASIC 10 and the digital ASIC 12. The housing member 19 including a lid member 19a comprises a material, such as aluminum or an aluminium alloy, having a light shielding characteristic.

The lid member 19a which is a part of the housing member 19 is removably attached to one end of the housing member 19. A unit joint FPGA (Field Programmable Gate Array, hereinafter referred to as FPGA 17), connectors 18, 24, 25, and C2 are provided in the lid member 19a. The signal wirings (not shown) of a plurality of connectors C2 are connected to FPGA 17, and power wirings (not shown) of these connectors C2 are connected to the connector 25. The signal wiring (not shown) of the connector 18 is connected to FPGA 17, and the power wiring (not shown) of the connector 18 is connected to the connector 24. The voltage regulator device 21 is connected to the connector 24 and the connector 25.

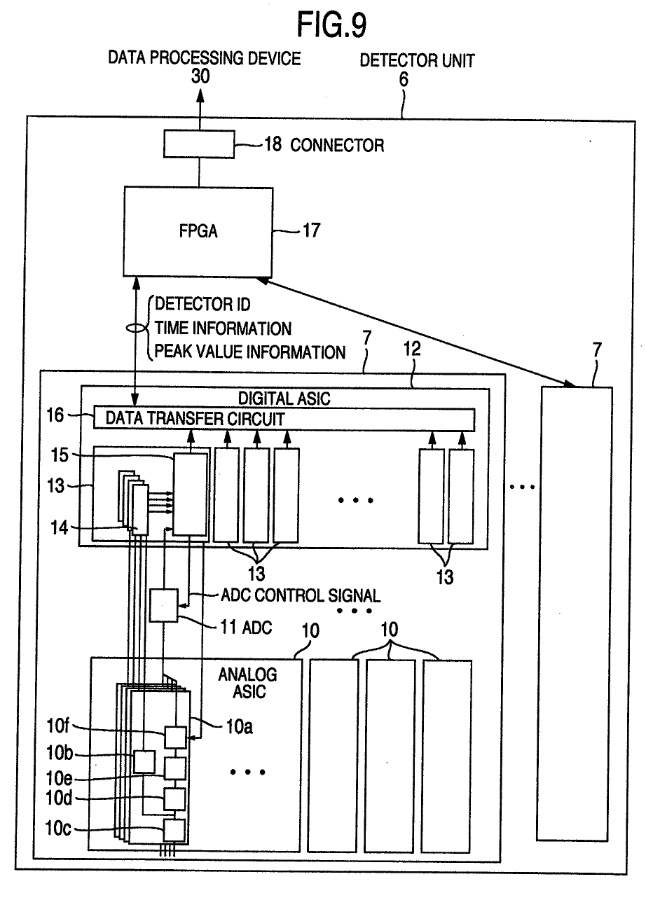

FIG. 9 is a view showing a detailed configuration of the module board. The detector 9, the analog ASIC 10, the analog digital converter (ADC 11), and the digital ASIC 12, and the connector C1 are connected in this order with signal wirings.

One analog ASIC 10 includes a plurality of analog signal processing circuits (analog signal processing devices) 10a. The analog signal processing circuit 10a having a fast system and a slow system is provided for each detector 9. The fast system has a timing pickoff circuit 10b which outputs a timing signal for identifying a detection time of a γ ray. In the slow system, a polarity amplifier (linear amplifier) 10d, a bandpass filter (waveform shaping device) 10e, and a peak hold circuit (peak value holding device) 10f are connected and provided in this order for the purpose of determining the peak value of a detected γ ray. The analog signal processing circuit 10a includes a charge amplifier (preamplifier) 10c which is connected to the timing pickoff circuit 10b and the polarity amplifier 10d. The charge amplifier 10c is connected to one detector 9.

The digital ASIC 12 comprises a plurality of packet data generation devices 13 including a plurality of time determination circuits (time information generation devices) 14 and one packet data generator 15, and a data transfer circuit (data source) 16 as shown in FIG. 9, and is an LSI of these. All the digital ASICs 12 provided in the device for positron emission computed tomography 1 receive a clock signal from a 500 MHz clock generator (not shown), and are synchronously operating. A clock signal input to the digital ASIC 12 is input to each of the time determination circuits 14 in the packet data generation device 13. Each of the time determination circuits 14 in the packet data generation device 13 is connected to the timing pickoff circuit 10b in the separate analog signal processing circuit 10a.

The detector 9 which detected an incident y ray outputs a γ ray detection signal. The detector 9 outputs a first γ ray detection signal when this γ ray is the first γ ray, and outputs a second γ ray detection signal when this γ ray is the second γ ray. In the followings, if described as a γ ray without discriminating the first γ ray from the second γ ray, it means both of the γ rays, and if described as a γ ray detection signal without discriminating the first γ ray detection signal from the second γ ray detection signal, it means both of the γ ray detection signals. The γ ray detection signal output from the detector 9 is amplified by the charge amplifier 10c and the polarity amplifier 10d. The amplified γ ray detection signal is input to the peak hold circuit 10f through the bandpass filter 10e. The peak hold circuit 10f holds the peak value of a γ ray detection signal.

A peak value signal output from the peak hold circuit 10f is converted to a digital signal by ADC 11, and input to the packet data generator 15. The timing pickoff circuit 10b receives a γ ray detection signal amplified by the charge amplifier 10c, and outputs a timing signal indicative of a timing when the γ ray was detected. This timing signal is input to the corresponding time determination circuit 14. The time determination circuit 14 determines the detection time of a γ ray based on a clock signal when the timing signal was received, and outputs detection time information.

Upon receipt of the detection time information, the packet data generator 15 identifies a detector ID of the detector 9 corresponding to the time determination circuit 14 which output this detection time information. The packet data generator 15 generates packet information which is digital information including detection time information, detector ID information (detector positional information), and peak value information (energy information of a γ ray detection signal) corresponding to one detector 9. This packet information is input to the data transfer circuit 16. The signal wiring (not shown) connected to the data transfer circuit 16 is connected to the signal wiring which is connected to the connector C2 via the connector C1. Note that the power wiring connected to the connector C2 is connected to the power wiring (not shown) connected to the connector C1. The latter power wiring is arranged in the board member 8, and is connected to each element, such as the detector 9 and the timing pickoff circuit 10b, provided in the module board 7.

The data transfer circuit 16 of each module board 7 arranged in one detector unit 6 respectively outputs packet information to FPGA 17 of the detector unit 6. Each packet information output from each FPGA 17 of all the detector units 2 provided in the imaging device 2 is transmitted to a signal discrimination device 31 of the data processing device 30 via the information transmission wiring (not shown) connected to each connector 18.

FIG. 6 is a vertical cross-sectional view of the imaging device 2 shown in FIG. 1. As shown in FIG. 6, the imaging device 2 further comprises a γ ray source device (radiation source device 48), a radiation source revolving device 54, and a radiation source linear-moving device 53. An annular rear end shield 60 is attached to a later-described rotating member 56. The detector unit 6 is arranged between the front end shield 5 and the rear end shield 60 in the axis direction of the imaging device 2. The front end shield 5 and the rear end shield 60 are radiation shielding bodies.

The radiation source revolving device 54 includes a rotation drive device 55 (e.g., motor), the rotating member 56, an annular gear member 57, a shaft supporting member 58, and a rolling member 59. The rotating member 56 is arranged at a rear end part of the housing 45. A part of the rotating member 56 is attached to the rolling member 59 (e.g., thrust bearing), which is arranged between the housing 45 and the rear end shield 60 and attached to a wall body 45. The rolling member 59 supports the rotating member 56. The rotation drive device 55 is attached to the housing 45. A gear provided in the rotation axis of the rotation drive device 55 engages with the gear member 57, which surrounds the peripheral part of the rotating member 56 and is attached to the rotating member 56. The rear end shield 60 rotates along with the rotating member 56. An encoder 63 attached to the rotation drive device 55 is connected to the rotation axis of the rotation drive device 55.

The radiation source device 48 includes the radiation source 26 which is a γ ray source, a radiation source supporting shaft 49, and a holding member 50. The radiation source 26 is attached to one end of the radiation source supporting shaft 49, and the holding member 50 is attached to the other end of the radiation source supporting shaft 49. The radiation source supporting shaft 49 is arranged so as to be parallel to the central axis (central axis of the measurement space R) Z of the imaging device 2. The radiation source 26 is arranged nearer to the central axis Z side, i.e., between the detector unit 6 and the bed 27, than the detector unit 6. The radiation source supporting shaft 49 extends through the shaft supporting member 58 mounted to the rotating member 56. The shaft supporting member 58 is a radiation shielding body, and seals one end part of a radiation source housing 61 formed inside the rotating member 56. A notch 60a for the radiation source 26 housed in the radiation source housing 61 to pass therethrough is provided in the rear end shield 60. Radiation source shields 64 and 65 which are radiation shielding bodies are provided. The radiation source shield 64 is arranged facing the notch 60a, and the radiation source shield 65 is arranged facing the position of the radiation source housing 61, respectively.

The radiation source 26 includes a radioisotope which emits a second γ ray of an energy different from that of the first γ ray. As this radioisotope, cesium 137 which emits a γ ray of 662 keV is used. In place of cesium 137, cobalt 57 (which emits the second γ ray of 122 keV), technetium 99m (which emits the second γ ray of 140 keV), tellurium 123m (which emits the second γ ray of 159 keV), cerium 139 (which emits the second γ ray of 166 keV), gadolinium 153 (which emits the second γ ray of 153 keV), americium 241 (which emits the second γ ray of 57 keV), and the like may be used. An X-ray source which is another radiation source may be used.

The radiation source linearly-moving device 53 comprises a moving device 51 and a guide member 52. The guide member 52 extends in the axis direction of the imaging device 2, and is attached to a side face of the rear end of the housing 45. The moving device 51 moves in the axis direction along the guide member 52, and has a groove 62 into which the holding member 50 is inserted. While the radiation source 26 is at the lowest position, that is, while the holding member 50 lies in the groove 62, the moving device 51 can move in this axis direction. With the movement of the moving device 51, the radiation source 26 is moved between the front end shield 5 and the shaft supporting member 58 in the axis direction of the imaging device 2. The radiation source 26 is housed in the radiation source housing 61 while T measurement is not performed. When performing T measurement, the rotating member 57 is rotated by driving the rotation drive device 55, and thereby the holding member 50 comes out of the groove 62 and the radiation source 26 revolves around the patient 29 lying on the bed 27.

FIG. 4 is a detailed configuration diagram of the device for positron emission computed tomography shown in FIG. 3. As shown in FIG. 4, the data processing device 30 comprises the signal discrimination device 31, a coincidence counting device 32, a second radiation processing device 34 (transmission data processing device), a storage device 35, a first tomographic image generation device 36 (emission image information generation device), a second tomographic image generation device 37 (transmission image information generation device), a motion compensation device 38, a radiation source position detection device 41, a motion phase information acquisition device 43, a phase information adding device 44, and a radiation source rotation control device 42. These devices comprises a computer, a circuit board, and the like.

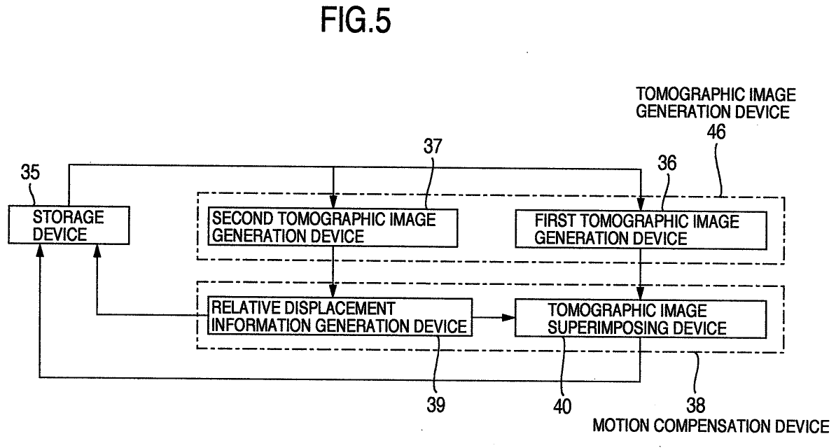

FIG. 5 is a detailed configuration diagram of the tomographic image generation device 46 and the motion compensation device 38 shown in FIG. 4. As shown in FIG. 5, the tomographic image generation device 46 includes the first tomographic image generation device 36 (emission image information generation device) and the second tomographic image generation device 37 (transmission image information generation device). As shown in FIG. 5, the motion compensation device 38 also includes a relative displacement information generation device 39 and a tomographic image superimposing device 40. The motion compensation method of FIG. 1 is executed by the tomographic image generation device 46 and the motion compensation device 38 of FIG. 5. The first tomographic image generation device 36 generates the emission images E₁, E₂, and E₃ of FIG. 1 based on the information of E measurement of the storage device 35. The second tomographic image generation device 37 generates the transmission images T₁, T₂, and T₃ of FIG. 1 based on the information of T measurement of the storage device 35. The relative displacement information generation device 39 generates F₂₁ and F₃₁ which are relative displacement information of FIG. 1. The tomographic image superimposing device 40 superimposes E₂ and E₃ onto E₁ of the reference phase of FIG. 1 using F₂₁ and F₃₁.

In FIG. 4, the signal discrimination device 31 is connected to the respective connector 18 of each detector unit 6, and is further connected to the coincidence counting device 32 and the second radiation processing device 34, respectively. The radiation source position detection device 41 is connected to the encoder 63, the second radiation processing device 34, and the radiation source rotation control device 42, respectively. The radiation source rotation control device 42 controls driving of the rotation drive device 55. An arithmetic-logic unit (not shown) is connected to the radiation source rotation control device 42, a respiration monitoring device 28, and a cardiac beat measuring device 67, respectively. The phase information adding device 44 is connected to the storage device 35, and acquires the pieces of information from the coincidence counting device 32, the second radiation processing device 34, and the motion phase information acquisition device 43. The mutual pieces of information of the devices in the data processing device 30 may be acquired directly between the devices without through the storage device 35. The motion phase information acquisition device 43 is connected to the respiration monitoring device 28 and the cardiac beat measuring device 67, respectively. The storage device 35 is connected to the first tomographic image generation device 36, the second tomographic image generation device 37, the relative displacement information generation device 39, the tomographic image superimposing device 40, the phase information adding device 44, and an information output device 66, respectively. The information output device 66 is connected to the display device 33. The relative displacement information generation device 39 is connected to the second tomographic image generation device 37 and the tomographic image superimposing device 40, respectively. The tomographic image superimposing device 40 is connected to the first tomographic image generation device 36.

The respiration monitoring device 28 is a device for monitoring breath temperature of the patient 29 and mounted on the face of the patient 29. The motion phase information acquisition device 43 receives information on the breath temperature measured by the respiration monitoring device 28, and calculates, as the respiration cycle, an interval between time points when the breath temperature becomes a peak at a timing that the respiration cycle changes from inhalation to exhalation. As the respiration monitoring device, an infrared camera or an optical stereoscopic camera for measuring a displacement of the chest skin of the patient 29 can be used. When the infrared camera or the optical stereoscopic camera is used, the corresponding camera attached to a supporting member, such as a tripod, is arranged at a position where the patient 29 can be imaged near the imaging device 2. In this case, the motion phase information acquisition device 43 analyzes a displacement waveform of the chest skin based on image information input from the infrared camera or the optical stereoscopic camera, and calculates the respiration cycle. Alternatively, a device capable of monitoring the respiration is separately prepared, and a signal output from this device, the signal providing a clue to the phase information analysis, may be input to the motion phase information acquisition device 43.

The cardiac beat measuring device 67 is mounted to the arm of the patient 29, measures the cardiac beat of the patient 29 and outputs the cardiac beat information measured by the motion phase information acquisition device 43.

The PET examination using the PET device of this embodiment and the generation of image information based on the information obtained by this examination will be described in detail.

An operator (a doctor or a radiological technician) inputs the information necessary for PET examination from an input device (not shown) provided in an operator console (not shown). This input information includes information concerning the patient 29 and PET pharmaceuticals, time information (e.g., 12 minutes) of PET examination, and furthermore information for respiratory motion compensation and information for cardiac beat compensation. For example, the information of respiratory compensation includes 8 which is the number of the first motion phases per respiration cycle (the number of respiratory motion phases), and 4 which is the number of the second motion phases per cardiac cycle (the number of cardiac beat phases). The operator further inputs an examination start command from this input device. Upon receipt of this examination start command, a bed driving device (not shown) moves the bed 27, on which the patient 29 administered with the PET radiopharmaceuticals is lying, to its longitudinal direction to insert the patient 29 in the measurement space R. The patient 29 is positioned at a predetermined position in the axis direction in the measurement space R.

Upon receipt of the above-described examination start command, the respiration monitoring device 28 starts the monitoring of respiration of the patient 29. Upon receipt of the above-described examination start command, the cardiac beat measuring device 67 starts the monitoring of cardiac beat of the patient 29. The measuring information of the respiration monitoring device 28, i.e., the measured breath temperature value of the patient 29, and the cardiac beat information measured by the cardiac beat measuring device 67 are input to the arithmetic-logic unit of the data processing device 30 and the motion phase information acquisition device 43. The arithmetic-logic unit calculates the respiration cycle based on the measured breath temperature value, and calculates the cardiac cycle based on the cardiac beat information. The arithmetic-logic unit stores the calculated respiratory waveform information of each respiration cycle and the calculated cardiac beat waveform information of each cardiac cycle into the storage device 35. The arithmetic-logic unit calculates the rotational cycle of the radiation source 26 serving as the γ ray source in which cycle the phase coverage of cardiac beat and respiration (i.e., the measured number of motion phases divided by the measured total number of motion phases) becomes the maximum in a short time. For example, if the value obtained by dividing a respiration cycle by a cardiac cycle is not a natural number, the rotational cycle of the radiation source 26 is shifted slightly from an integer multiple of the respiration cycle or such operation is performed to calculate the rotational cycle. The arithmetic-logic unit outputs the calculated rotational cycle of the radiation source 26 as the rotational cycle setting value to the radiation source rotation control device 42. When the respiration cycle of the patient 29 needs to be finely adjusted based on the calculated rotational cycle setting value, the arithmetic-logic unit will output voice guide information to a respiratory guide device 68. This guide information is output as a voice from the respiratory guide device 68 and is transmitted to the patient 29.

The information output device 66 outputs the respiratory waveform information for each respiration cycle of the patient 29 and the cardiac beat waveform information for each cardiac cycle, which are read from the storage device 35, to the display device 33. The outputting of the relevant information to the display device 33 by the information output device 66 is performed in response to a request command from the operator which is input from the above-described input device. When the respiratory waveform information and cardiac beat information of the patient 29 displayed on the display device 33 become stable, the operator inputs a T measurement start command to start T measurement, to the input device. The respiratory waveform information and cardiac beat information become stable in several minutes. T measurement is performed in parallel with E measurement while the E measurement is being performed.

The T measurement start command is input to the motion phase information acquisition device 43, a radiation source linear-movement control device (not shown), and the radiation source rotation control device 42, respectively, from the input device.

Upon receipt of the T measurement start command, the motion phase information acquisition device 43 calculates the respiration cycle based on an input measured temperature value, and divides the respiratory waveform information of each calculated respiration cycle on the time-axis, based on the number of the first motion phases input from the input device. For each first motion phase (respiration phase) divided on the time-axis based on the number of the first motion phases (e.g., 5), first time information is provided. Moreover, the motion phase information acquisition device 43 calculates the cardiac cycle based on the input cardiac beat information, and then divides the cardiac beat waveform information of each calculated cardiac cycle on the time axis, based on the number of the second motion phases (e.g., 4) input from the input device. For each second motion phase (cardiac beat phase) divided on the time axis based on the number of the second motion phases, second time information is provided. For example, when the calculated respiration cycle is 3 seconds, the time width of one first motion phase is 0.6 seconds because the number of the input first motion phases is 5. For every five first motion phases divided for each 0.6 seconds, the first time information is provided. This processing is repeated for each respiration cycle. The information of each first motion phase provided with the first time information for each respiration cycle is input to the phase information adding device 44. For example, when the calculated cardiac cycle is 0.8 seconds, the time width of the second one motion phase is 0.2 seconds because the number of the input second motion phases is 4. For each four second motion phases divided for each 0.2 seconds, the second time information is provided. This processing is repeated for each cardiac cycle. The information of each second motion phase provided with the second time information for each cardiac cycle is input to the phase information adding device 44.

Upon receipt of the T measurement start command, the control device for controlling the radiation source linearly-moving device 53 outputs a drive command to the moving device 51. Upon receipt of the drive command, the moving device 51 moves toward the housing 45 along the guide member 52. The radiation source supporting shaft 49 engaged with the moving device 51 moves toward the front end shield 5. Thus, the radiation source 26 moves outward from the radiation source housing 61, and is set at a predetermined position between the front end shield 5 and the rear end shield 60.

Upon receipt of the T measurement start command, the radiation source rotation control device 42, after the radiation source 26 is set at the above-described predetermined position, controls the rotation of the rotation drive device 55 so that the rotational cycle of the radiation source 26 becomes the rotational cycle setting value input from the arithmetic-logic unit. The rotational force of the rotation drive device 55 is transmitted to the rotating member 56 via the gear member 57, and rotates the rotating member 56. The radiation source 26 rotates along with the rotating member 56, and revolves around the patient 29 so as to meet the rotational cycle setting value. The second γ ray of 662 keV emitted from the radiation source 26 is irradiated from the perimeter to the patient 29 lying on the bed 27 as the radiation source 26 revolves. The T measurement is performed while revolving the radiation source 26.

For example, when the revolution of the radiation source 26 is started, an operator inputs a data acquisition start command to the above-described input device. When this data start command is input to the signal discrimination device 31, the signal discrimination device 31 starts to receive each packet information output from each detector unit 6.

Generation of the respective pieces of packet information to be input to the signal discrimination device 31, in E measurement and T measurement, is described.

While the patient 29 is being inserted in the measurement space R, all the detectors 9 surround the perimeter of the patient 29. Under this state, E measurement is implemented. The paired γ rays (a pair of first γ rays) generated in the annihilation of a positron caused by the PET radiopharmaceuticals accumulated at a malignant tumor-affected part are incident upon a pair of detectors 9 positioned in about 180° opposite directions of the imaging device 2, and are detected by these detectors 9. The detector 9 which detected the first γ ray outputs the first γ ray detection signal. Upon receipt of this first γ ray signal, the timing pickoff circuit 10b of the analog signal processing circuit 10a outputs a timing signal, and the peak hold circuit 10f outputs a peak value signal. Upon receipt of this timing signal, the time determination circuit 14 generates detection time information of the first γ ray which is determined based on this timing signal, as described above. Upon receipt of the peak value information converted to a digital signal by ADC 11 and the detection time information, the packet data generator 15 generates packet information (hereinafter, referred to as first packet information) with respect to the detected first γ ray. This first packet information includes the detection time information, detector ID information, and peak value information with respect to the first γ ray. The first packet information obtained by E measurement is input to the signal discrimination device 31.

The second γ ray emitted from the radiation source 26 and passing through the patient 29 in T measurement is detected by the detector 9. The second γ ray detection signal output from the detector 9 which detected the second γ ray is processed by the analog signal processing circuit 10a and the packet data generation device 13, as with the first γ ray detection signal. The packet data generator 15 generates packet information (hereinafter, referred to as second packet information) with respect to the detected second γ ray. This second packet information includes the detection time information, detector ID information, and peak value information with respect to the second γ ray. The second packet information obtained in T measurement is also input to the signal discrimination device 31.

When T measurement is finished, an operator inputs a T measurement finish command from the above-described input device. Upon receipt of the T measurement finish command, the radiation source rotation control device 42 outputs a stop control command to the rotation drive device 55, and stops the rotation drive device 55 when the radiation source 26 serving as a γ ray source reaches the lowest position. Under this state, the holding member 50 is positioned in the groove 62 of the moving device 51. Upon receipt of the T measurement finish command, the radiation source linear-movement control device controls so that the moving device 51 may move away from the housing 45. Since the moving device 51 moves so as to move away from the housing 45, the radiation source 26 is housed in the radiation source housing 61, and the irradiation of the second γ ray onto the patient 29 is stopped.

Since E measurement and T measurement are being performed in parallel after a data start command is input, the signal discrimination device 31 receives both of the first packet information and the second packet information output from each detector unit 6, and discriminates these pieces of packet information based on an energy of the detected γ ray, i.e., the peak value information. The signal discrimination device 31 outputs to the coincidence counting device 32 the peak value information corresponding to the energy of the first γ ray, i.e., the first packet information including the peak value information of a range corresponding to the energy of 450 to 550 keV, for example. The second packet information including the peak value information corresponding to the energy of the second γ ray, i.e., the peak value information of a range corresponding to, for example, the energy of 570 to 650 keV, is output to the second radiation processing device 34. Switching of the output destinations of these pieces of packet information is performed by switching a changing-over switch (not shown) provided in the signal discrimination device 31. In providing time information in the case where E measurement and T measurement are performed in parallel, unless synchronized, the start time of a respiration cycle and the detection time of a γ ray determined by the time determination circuit 14 will deviate from each other, posing a problem in generating E image information and T image information for each motion phase in the first tomographic image generation device 36 and the second tomographic image generation device 37. The synchronization method of E gated-acquisition and T gated-acquisition is described. A packet including time information is sent from the detector unit 6 to the data processing device 30. The motion phase information acquisition device 43 acquires the packet including time information via the storage device 35 in real time. The phase information adding device 44 obtains the time information from the motion phase information acquisition device 43.

The coincidence counting device 32 performs coincidence counting using the detection time information and the detector ID which are obtained based on the first γ ray detection signal, and identifies a pair of detectors 9 which detected a pair of first γ rays generated due to the annihilation of one positron. The coincidence counting device 32 outputs each detector ID information and detection time information contained in the first packet information of the identified pair of detectors 9, to the phase information adding device 44. Each detector ID information and detection time information obtained by coincidence counting are referred to as the first detection information.

The radiation source position detection device 41 receives an output signal (rotation angle information of the rotation drive device 55) of the encoder 63, and calculates the positional information (hereinafter, referred to as radiation source positional information) of the revolving radiation source 26 based on this output signal. The radiation source positional information is input to the second radiation processing device 34 and the radiation source rotation control device 42, as the feedback information. The radiation source rotation control device 42 controls the rotation drive device 55 based on the rotational cycle setting value and the fed-back radiation source positional information.

The second radiation processing device 34 outputs the detector ID information and detection time information of the detector 9 which detected the second γ ray, the detector ID information and detection time information being contained in the second packet information, and the added radiation source positional information to the phase information adding device 44. The detector ID information, detection time information, and radiation source positional information output from the second radiation processing device 34 are referred to as the second detection information.

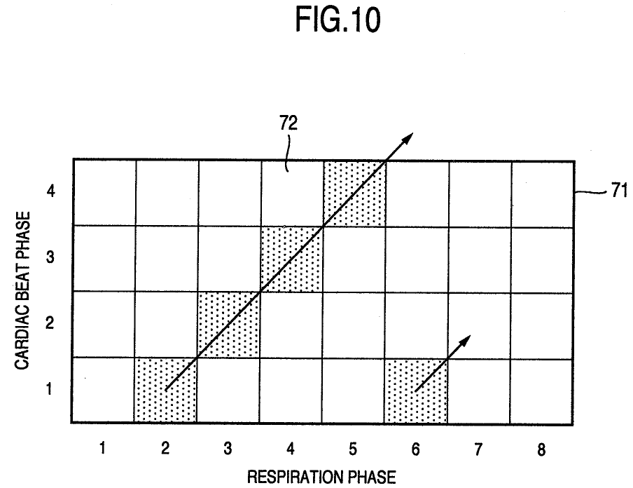

By use of the first time information provided to the first motion phase information and the second time information provided to the second motion phase information, the phase information adding device 44 provides the corresponding first motion phase information to the first detection information input from the coincidence counting device 32, and stores the resultant information into the storage device 35. That is, the phase information adding device 44 provides the first detection information with the first motion phase information of the first time information and the second motion phase information of the second time information which match the detection time information contained in the first detection information. Note that, this phase information addition processing may be performed anew after the first detection information, first motion phase information, and second motion phase information are stored into a temporary storage area in the phase information adding device 44 or into the storage device 35 once. The above-described first detection information is stored in a two-dimensional table (see FIG. 10) 71 in the storage device 35.

FIG. 10 is an explanatory view showing the two-dimensional table in the storage device 35 shown in FIG. 4. The horizontal axis of FIG. 10 represents the first motion phase (respiration phase), and the vertical axis represents the second motion phase (cardiac beat phase). Based on the first and second motion phase information, the first detection information is stored in a motion phase (hereinafter, referred to as an interval area 72) at an intersection between the corresponding first motion phase and the corresponding second motion phase. In this manner, each first detection information output from the coincidence counting device 32 is sequentially stored in the corresponding interval area 72 in the two-dimensional table 71.

By use of the first time information provided to the first motion phase information and the second time information provided to the second motion phase information, the phase information adding device 44 provides the second detection information input from the second radiation processing device 34 with the corresponding first motion phase information, and stores the resultant information in the storage device 35. That is, the phase information adding device 44 provides the second detection information with the first motion phase information of the first time information and the second motion phase information of the second time information which match the detection time information contained in the second detection information. Note that, this phase information addition processing may be performed anew after the second detection information, first motion phase information, and second motion phase information are stored into a temporary storage area in the phase information adding device 44 or into the storage device 35 once. The above-described first detection information is stored in the two-dimensional table 71 in the storage device 35. Based on the first and second motion phase information and the radiation source positional information contained in the second detection information, the first detection information is stored in the corresponding interval area 72. In this manner, each second detection information output from the second radiation processing device 34 is sequentially stored in the corresponding interval area 72 in the two-dimensional table 71.

Each interval area 72 of the two-dimensional table 71 corresponds to a time interval which is determined by individually combining each of a plurality of first motion phases obtained by dividing the respiration cycle of the patient 29, with each of a plurality of second motion phases obtained by temporally dividing the cardiac cycle.

The position of the interval area 72 in the two-dimensional table 71 is denoted by (m,n). Here, m represents the number of the second motion phases and n represents the number of the first motion phases. For example, in the two-dimensional table 71, an interval area (3,2) indicates the interval area 72 which is a motion phase at an intersection between the first motion phase of “3” and the second motion phase of “2.”

The second tomographic image generation device 37 reconstructs the respective T image information using the detector ID information and radiation source positional information contained in the second detection information stored in the respective interval area 72, for each interval area 72 of the two-dimensional table 71. To describe the two-dimensional table 71 as an example, T image information T_{(1,1)}, . . . , T image information T_{(2,1)}, . . . , T image information T_{(4,1)}, . . . , T image information T_{(4,5)} in each of an interval area (1,1), . . . , an interval area (2,1), . . . , an interval area (4,1), . . . , an interval area (4,5) are generated, respectively. These pieces of T image information correspond to the T image information T₁, T₂, T₃, . . . shown in FIG. 1. The second tomographic image generation device 37 stores the generated T image information of each second area into a corresponding interval area (m, n) in the two-dimensional table 71, respectively.

The first tomographic image generation device 36 reconstructs the respective E image information using each detector ID information which is contained in the first detection information stored in the respective interval area 72, the each detector ID information being obtained by coincidence counting, for each interval area 72 of the two-dimensional table 71. To describe the two-dimensional table 71 as an example, E image information E_{(1,1)}, . . . , E image information E_{(2,1)}, . . . , E image information E_{(4,1)}, . . . , E image information E_{(4,5)} in each of the interval area (1,1), . . . , the interval area (2,1), . . . , the interval area (4,1), . . . , the interval area (4,5) are generated, respectively. These pieces of E image information correspond to the E image information E₁, E₂, E₃, . . . shown in FIG. 1. The first tomographic image generation device 36 stores the generated E image information of each interval area into the corresponding interval area (m,n) in the two-dimensional table 71, respectively.

In reconstructing E image information in a certain interval area (m, n) (e.g., interval area (2,3)), based on T image information T_{(m, n)} concerning the transmissivity of the second γ ray, the T image information T (m, n) being reconstructed in the interval area (m, n) (e.g., the second area (2,3)), attenuation correction with respect to the first detection information is performed to reconstruct E image information E_{(m, n)}.

The first tomographic image generation device 36 and the second tomographic image generation device 37 generate E image information and T image information for each first area of the two-dimensional table 71, and E image information and T image information for each second area of the two-dimensional table 71, respectively, using a tomographic image reconstruction algorithm, for example, such as the filtered back projection method. E image information and T image information are tomographic image information, respectively. In this embodiment, the identity of the conformation of the patient 29 is guaranteed by parallel measurement (substantially coincidence measurement) of E measurement and T measurement. For this reason, in this embodiment, artifacts and quantitative degradation associated with a positional deviation in E gated-acquisition and T gated-acquisition in attenuation correction are significantly reduced as compared with those in the conventional E gated-acquisition.

When three-dimensional imaging is performed, the above-described T image information and E image information are reconstructed using a Fourier re-binning method or a three-dimensional successive approximation reconstructing method described in IEEE Transactions on Medical Imaging, Vol. 18, P. 657, for example.

A specific processing in this embodiment is described, in which the pieces of T image information for each motion phase and the pieces of E image information for each motion phase are nonlinearly superimposed onto each other, respectively, as described using FIG. 1. These processings are performed by the motion compensation device 88. The relative displacement information generation device 39 receives T image information of each interval area stored in the two-dimensional table 71 of the storage device 35 (e.g., T image information T_{(1,1)}, . . . , T image information T_{(4,5)}, and the information of each interval area (e.g., the interval area (1,1), . . . , the interval area (4,5)). The relative displacement information generation device 39, targeting at one set of certain respiration cycle and certain cardiac cycle, superimposes T image information T_{(1,2)}, . . . , T image information T_{(2,1)}, . . . , T image information T_{(4,1)} . . . , and T image information T_{(4,5)} in each of an interval area (1,2), . . . , an interval area (2,1), . . . , an interval area (4,1), . . . , an interval area (4,5) onto the T image information T_{(1,1)} of the interval area (1,1) (reference interval area), and generates T superimposed image information with respect to one respiration cycle and one cardiac cycle. This nonlinear superimposing processing of image information is performed using an algorithm called non-rigid image registration method described in IEEE Transactions on Medical Imaging, Vol. 18, PP. 712-720. With the superimpose of these pieces of T image information, the respective relative displacement information, i.e., transformation-matrix information (corresponding to transformation-matrix information Fⱼ₁ shown in FIG. 1) between T image information of the reference interval area (e.g., T image information T_{(1,2)}) and the respective T image information of the other interval areas (e.g., T image information T_{(1,2)}, . . . , T image information T_{(4,1)}, . . . , T image information T_{(4,5)}) can be obtained. These pieces of relative displacement information are stored in the storage device 35. In the superimposing processing on T image information T_{(1,1)} of this reference interval area, by use of each T image information of each of the other interval areas in a set of one respiration cycle and one cardiac cycle, the pixel value of each pixel at spatially the same position of this T image information is added up. This processing is performed on all the pixels of T superimposed image information. T superimposed image information having such pixel value information is stored in the storage device 35. T superimposed image information generation processing described above is performed for each set of respiration cycle and cardiac cycle. The relative displacement information generation device 39 generates the respective T superimposed image information and each transformation-matrix information in the reference phase (reference interval area) for each set of respiration cycle and cardiac cycle within the period of PET examination.

Furthermore, by use of each T superimposed image information with respect to the reference interval area generated for each set of respiration cycle and cardiac cycle, the relative displacement information generation device 39 performs a process of adding up the pixel value of each pixel at spatially the same position of these pieces of image information, onto all the pixels of one piece of T superimposed image information. The final T superimposed image information (i.e., statistical-noise-suppressed motion-compensated image) obtained by adding up such pixel values has high statistical accuracy, resulting in clearer image information. The final T superimposed image information is stored in the storage device 35. The tomographic image superimposing device 40 receives E image information of each interval area stored in the two-dimensional table 71 of the storage device 35 (e.g., E image information E_{(1,1)}, . . . , E image information E_{(4,5)} and information of each interval area (e.g., the interval area (1,1), . . . , the interval area (4,5)). The tomographic image superimposing device 40, in the above-described set of one certain respiration cycle and one certain cardiac cycle, nonlinearly superimposes E image information E_{(1,2)}, image information E_{(2,1)}, . . . , E image information E_{(4,1)}, . . . , E image information E_{(4,5)} in each of the interval area (1,2), . . . , the interval area (2,1), . . . , the interval area (4,1), . . . and the interval area (4,5), by use of the respective transformation-matrix information (corresponding to transformation-matrix information Fⱼ₁ shown in FIG. 1) calculated by the relative displacement information generation device 39, onto the E image information E_{(1,1)} of the interval area (1,1) (reference interval area) and generates E superimposed image information with respect to the set of one respiration cycle and one cardiac cycle, as in the processing concept shown in FIG. 1. This nonlinear superimposing processing of image information is performed using the algorithm called non-rigid image registration method described above. By use of each E image information of each interval area, the tomographic image superimposing device 40 performs the process of adding up the pixel value of each pixel at spatially the same position of these pieces of E image information onto all the pixels of E image information of the reference interval area. The obtained E superimposed image information is stored in the storage device 35. The E superimposed image information generation processing described above is performed for each set of respiration cycle and cardiac cycle within the period of PET examination.

Furthermore, by use of each E superimposition image information with respect to the reference interval area generated for each set of respiration cycle and cardiac cycle, the tomographic image superimposing device 40 performs the process of adding up the pixel value of each pixel at spatially the same position of these pieces of E superimposed image information onto all the pixels of one piece of T superimposed image information. The final E superimposed image information (i.e., statistical-noise-suppressed motion-compensated image) obtained by adding up such pixel values has high statistical accuracy, resulting in clearer image information. The final E superimposed image information is stored in the storage device 35.

The information output device 66, among each E image information and each T image information of each interval area, each E superimposed image information and each T superimposed image information for each set of respiration cycle and cardiac cycle, and the final E superimposed image information and the final T superimposed image information, reads the respective image information alone or two or more kinds thereof altogether from the storage device 35, and outputs the same to the display device 33. The display device 33 displays the input image information. The image information to be displayed in the display device 33 is read from the storage device 35 by the information output device 66, based on an image information display command which an operator inputs from the input device of the operator console.

In this embodiment, by use of relative displacement information, i.e., transformation-matrix information, obtained by superimposing T image information of other motion phase areas (other interval areas) onto T image information of one motion phase area (e.g., reference interval area) specified by one certain first motion phase and one certain second motion phase, E image information of the above-described other motion phase areas (other interval areas) are superimposed onto E image information of the one motion phase area (reference interval area). Accordingly, all the E image information of each motion phase area in a set of one respiration cycle and one cardiac cycle can be superimposed. For this reason, also in a region influenced by a respiratory motion and a cardiac beat, clearer E image information (the final E superimposed image information) can be obtained in a shorter time. According to the clear E image information (the final E superimposed image information) obtained by this embodiment, diagnosis on a malignant tumor present in a region which moves under the influence of respiration and cardiac beat can be performed accurately. The statistical accuracy expected for this E image information in this embodiment corresponds to E measurement for 12 minutes. Usually, E measurement for 12 minute can satisfy the statistical accuracy sufficiently. Moreover, in this embodiment, since a respiratory motion and a cardiac beat are compensated by each T image information in each motion phase area, this final E superimposed image information results in clear image information with less blurring and high quantitativity. Moreover, in this embodiment, since a positional deviation of the patient 29 will not occur in T measurement and E measurement at the time of attenuation correction, the artifact associated with this positional deviation will not occur, either. During the period of PET examination in this embodiment, the patient 29 can continue quiet respiration and does not need to stop breathing.

The reason why relative displacement information obtained based on each T image information of a motion phase area as described above can be utilized for superimpose of E image information of a different motion phase area is that E measurement and T measurement are performed in parallel and that the first γ ray and the second γ ray are detected with one detector 9. One detector 9 often outputs the first γ ray detection signal and the second γ ray detection signal, with a temporal deviation from each other. The first packet information and the second packet information generated based on these γ ray detection signals can be easily discriminated by the signal discrimination device 31 based on the energy of the γ ray detection signal. That is, now that the signal discrimination device 31 receives a peak value corresponding to the energy of a γ ray detection signal which is obtained by the peak hold circuit (peak value generation device) 10f, the signal discrimination device 31 can easily discriminate the first packet information and the second packet information which are obtained by the peak hold circuit (peak value generation device) 10f.

If necessary, in adding up pixel values of E image information of other motion phase areas to a pixel value of E image information of the reference phase area (reference interval area), if a processing is performed, in which the pixel value is adjusted taking into consideration the degree of expansion and contraction of nonlinearly distorted E image information and is added up, then the quantitativity of the degree of accumulation of the PET radiopharmaceuticals can be improved further.

In one or more display devices 33 provided in the operator console, E image information and T image information of the same motion phase area or E superimposed image information and T superimposed image information of the reference phase area can be displayed together with the final E superimposed image information. Such display allows evaluating the final E superimposed image information.

Note that, in the nuclear medicine diagnosis apparatus, a γ ray from a patient may be scattered within a certain radiation detector and absorbed by another radiation detector and thereby an energy may be provided to a plurality of radiation detectors. In such a case, whether a γ ray before being scattered is a γ ray from the radiopharmaceuticals administered to the subject P may be determined based on radiation detection information in two or more radiation detectors, and if so, the relevant γ may be processed as the effective signal. Hereinafter, such a method is called scattered-radiation processing. The nuclear medicine diagnosis apparatus includes a scattered-radiation processing means which identifies a plurality of radiation signals caused by the radiation rays scattered by a radiation detector, as one radiation signal based on the output signals output from the radiation detectors. Here, the scattered-radiation processing means may be either of the packet data generator 15 and FPGA 17. Moreover, the scattered-radiation processing means may be provided on the upstream side of data processing of the signal discrimination device 31 of the data processing device 30. Since the number of effective signals will increase due to the scattered-radiation processing, an accurate diagnosis image can be expected.

Note that, although an example of the device for positron emission computed tomography has been shown in the above-described embodiment, a SPECT device may be used, and a device capable of imaging the functional image of a living organ may be used. Moreover, this embodiment can be applied to not only a two-dimensional tomogram but a relationship between a three-dimensional functional image and structural image of a living organ. The image generation method described above is applicable to a computer captured image wherein a medical image is affected by the influence of a patient motion. That is, in order to obtain clear functional image information on a living organ in a short time, targeting at a region influenced by a patient motion, an image generation method of an image processing device is performed, the method comprising the steps of: generating first tomographic image information in a plurality of time intervals which are defined by individually combining a plurality of first phases obtained by temporally dividing a certain cycle with a plurality of second phases obtained by temporally dividing other cycle different from the certain cycle; generating second image information of a structure of a living organ imaged in the plurality of time intervals; superimposing the second image information of the other time interval onto the second image information of the one certain time interval among the plurality of time intervals and calculating relative displacement information between these pieces of second image information; and generating first superimposed image information by superimposing the first image information of the other time interval onto the first image information of the one certain time interval by use of the relative displacement information.

Note that, in the above-described embodiment, the number of partitions of a respiration cycle with respect to E image information and T image information is set to three or eight and the number of partitions of a cardiac cycle is set to four, however, a designated arbitrary number of partitions may be used. Moreover, as the partition of the time of a respiration cycle, an example of equal partitions has been shown, however, the partition of the time may not be equal partitions, and the time of a certain partition interval may be made shorter or longer than the time of other partition interval. For example, the time of an interval more affected by a patient motion is shortened and the time of an interval less affected by the patient motion is lengthened, so that image processing can be performed while discriminating the interval less affected by the patient motion from the interval more affected by the patient motion, and a clearer image can be obtained. This is also true of the partition of a cardiac cycle. When the time of a certain partition interval is made shorter or longer than the time of other partition interval, a superimposed image can be obtained by dividing by an added-up imaging time on the right side of Equation (3) instead of dividing by n. Moreover, in the case where the time of a certain partition interval is made shorter or longer than the time of other partition interval, if the length of each grid of the two-dimensional table of FIG. 10 is displayed in the unit of time, each grid becomes a rectangular instead of a square and accordingly a different relationship of a patient motion can be easily known.

As for the interval area determined by a set of respiration phase and cardiac beat phase, it is not necessary to use all the interval areas in superimposing, and some of the interval areas may be designated and used in superimposing. For example, among all the interval areas, an interval area including a time point of having finished to exhale the air and an interval area including a time point of having finished to inhale the air, which are interval areas less affected by a patient motion, may be designated to perform superimposing, thereby allowing for a quick measurement using images less affected by a patient motion. Moreover, an image emphasizing prevention of blurring in the motion of a cardiac beat can be obtained by designating a specific cardiac beat phase without designating a respiration phase. Likewise, if a specific respiration phase is designated but a cardiac beat phase is not designated, an image emphasizing prevention of blurring in the motion of respiration can be obtained. Moreover, by designating a specific respiration phase and a specific cardiac beat phase, an image emphasizing prevention of blurring in the both patient motions can be obtained. Moreover, by eliminating an image interval which is more affected by a patient motion and in which an index indicative of the degree of blurring exceeds a predetermined value, it is possible to improve the accuracy of an image while placing high priority on quick measurement.

Note that, in the above-described embodiments, an example has been shown, in which E image information serving as a functional image of a living organ and T image information serving as a structural image of the living organ are taken with one imaging device to synchronize with the cycle of a patient motion, however, it is also possible to synchronize with the cycle of a patient motion between a functional image of a living organ taken using a certain imaging device and a structural image of the living organ image taken using another imaging device. For example, by identifying the cycle of a functional image and identifying the cycle of a structural image, and then assigning an interval to be divided so that the starting points of the respective cycles and the lengths of the cycles may be aligned with each other, respectively, it is possible to synchronize a functional image with a structural image.

It should be further understood by those skilled in the art that although the foregoing description has been made on embodiments of the invention, the invention is not limited thereto and various changes and modifications may be made without departing from the spirit of the invention and the scope of the appended claims.

1. An image generation method of an image processing device, the method comprising the steps of:
generating first tomographic image information in a plurality of time intervals, which are defined by individually combining a plurality of first phases obtained by temporally dividing a certain cycle with a plurality of second phases obtained by temporally dividing other cycle different from the certain cycle;
generating second image information of a structure of a living organ imaged in the plurality of time intervals;
superimposing the second image information of other time interval onto the second image information of the one certain time interval among the plurality of time intervals, and calculating relative displacement information between these pieces of second image information; and
generating first superimposed image information by superimposing the first image information of the other time interval onto the first image information of the one certain time interval by use of the relative displacement information.

2. A tomographic image generation method of a computed tomography device, the method comprising the steps of:
based on a plurality of first information obtained from a plurality of first radiation detection signals which are output from a plurality of radiation detectors surrounding a bed upon incidence of a first radiation ray caused by radiopharmaceuticals, generating first tomographic image information in a plurality of time intervals, which are defined by individually combining a plurality of first phases obtained by temporally dividing a certain cycle with a plurality of second phases obtained by temporally dividing other cycle different from the certain cycle;
generating second tomographic image information in the time interval based on a plurality of second information obtained from a plurality of second radiation detection signals which are output from the plurality of radiation detectors upon incidence of a second radiation ray emitted from a radiation source;
superimposing the second tomographic image information of the other time interval onto the second tomographic image information of the one certain time interval among the plurality of time intervals, and calculating relative displacement information between these pieces of second tomographic image information; and
generating first superimposed tomographic image information by superimposing the first tomographic image information of the other time interval onto the first tomographic image information of the one certain time interval by use of the relative displacement information.

3. The tomographic image information generation method of a computed tomography device according to claim 2, wherein the first information and the second information are discriminated from each other by a difference in an energy between the first radiation detection signal and the second radiation detection signal, and wherein the first tomographic image information is generated using the discriminated first information while the second tomographic image information is generated using the discriminated second information.

4. The tomographic image information generation method of a computed tomography device according to claim 2, wherein the relative displacement information between the pieces of the second tomographic image information is nonlinear relative displacement information, and wherein the first superimposed tomographic image information is generated using the nonlinear relative displacement information.

5. The tomographic image information generation method of a computed tomography device according to claim 2, wherein the generation of the first tomographic image information of the time interval is performed by performing attenuation correction of the first information corresponding to this time interval using the second tomographic image information of this time interval.

6. The tomographic image information generation method of a computed tomography device according to claim 2, wherein the first information includes first detection time information of the first radiation ray and the second information includes second detection time information of the second radiation ray, and wherein the generation of the first tomographic image information for the each time interval is performed using the first information identified with the first detection time information, and the generation of the second tomographic image information for the each time interval is performed using the second information identified with the second detection time information.

7. The tomographic image information generation method of a computed tomography device according to claim 2, the method further comprising the steps of: detecting a position of the radiation source in a revolving direction around the bed; and using the detected plurality of radiation source positional information in generating the second tomographic image information.

8. A tomographic image information generation method of a computed tomography device, the method comprising the steps of:
generating a plurality of first information including first detection time information of a first radiation ray, the first detection time information being obtained from a plurality of first radiation detection signals which are output from a plurality of radiation detectors surrounding a bed upon incidence of the first radiation ray caused by radiopharmaceuticals, first positional information on the radiation detector from which the first radiation detection signal is output, and first energy information of the first radiation detection signal;
performing coincidence counting based on the detection time information contained in the plurality of first information, and identifying a pair of radiation detectors which output the first radiation detection signal within a setting time;
generating first tomographic image information in a plurality of time intervals, which are defined by individually combining a plurality of first phases obtained by temporally dividing a certain cycle with a plurality of second phases obtained by temporally dividing other cycle different from the certain cycle based on the first positional information and the first detection time information of the identified pair of radiation detectors;
generating a plurality of second information obtained from a plurality of second radiation detection signals which are output from the plurality of radiation detectors upon incidence of a second radiation ray emitted from a radiation source;
detecting a position of the radiation source in a revolving direction around the bed;
generating second tomographic image information for the each phase with respect to the plurality of phases based on the plurality of second information and the detected plurality of radiation source positional information;
superimposing the second tomographic image information of the other time interval onto the second tomographic image information of the one certain time interval among the plurality of time intervals, and calculating relative displacement information between these pieces of second tomographic image information; and
generating first superimposed tomographic image information by superimposing the first tomographic image information of the other time interval onto the first tomographic image information of the one certain time interval by use of the relative displacement information.

9. The tomographic image information generation method of a computed tomography device according to claim 8, wherein
the second information includes second detection time information of the second radiation ray, second positional information on the radiation detector from which the second radiation detection signal is output, and second energy information of the second radiation detection signal, wherein
the first information and the second information are discriminated from each other by the first energy information and the second energy information, and wherein
the coincident counting is performed using the first detection time information of the discriminated first information and the generation of the second tomographic image information is performed using the discriminated second information.

10. The tomographic image information generation method of a computed tomography device according to claim 2, wherein the radiation detector is a semiconductor radiation detector.

11. A computed tomography device, comprising:
a bed on which a subject is placed;
a radiation source revolving around the bed;
a plurality of radiation detectors arranged around the bed, which detect a first radiation ray caused by radiopharmaceuticals and emitted from the subject and output a first radiation detection signal, and detect a second radiation ray emitted from the radiation source and output a second radiation detection signal;
a first tomographic image generation device which, based on a plurality of first information obtained from the plurality of first radiation detection signals, generates first tomographic image information in a time interval defined by individually combining a plurality of first motion phases obtained by temporally dividing a respiration cycle of the subject with a plurality of second motion phases obtained by temporally dividing a cardiac cycle generate;
a second tomographic image generation device which generates second tomographic image information in the plurality of time intervals based on a plurality of second information obtained from the plurality of second radiation detection signals;
a relative displacement information generation device which superimposes the second tomographic image information of other time interval onto the second tomographic image information of the one certain time interval among the plurality of time intervals, and calculates relative displacement information between these pieces of second tomographic image information; and
a tomographic image information superimposing device which generates first superimposed tomographic image information by superimposing the first tomographic image information of the other time interval onto the first tomographic image information of the one certain time interval by use of the relative displacement information.

12. The computed tomography device according to claim 11, further comprising: a discrimination device which receives the first information and the second information, and discriminates the first information and the second information from each other based on a difference in an energy between the first radiation detection signal and the second radiation gland detection signal, wherein
the first tomographic image generation device generates the first tomographic image information using the discriminated first information; and
the second tomographic image generation device generates the second tomographic image information using the discriminated second information.

13. The computed tomography device according to claim 11, wherein the first tomographic image generation device generates the first tomographic image information of the time interval by performing attenuation correction of the first information corresponding to the time interval using the second tomographic image information of the time interval.

14. The computed tomography device according to claim 11, wherein
the first tomographic image generation device performs generation of the first tomographic image information for the each time interval using the first information which is identified with the first detection time information of the first radiation detection signal contained in the first information, and wherein
the second tomographic image generation device performs generation of the second tomographic image information for the each time interval using the second information which is identified with the second detection time information of the second radiation detection signal contained in the second information.

15. The computed tomography device according to claim 11, further comprising: a radiation source position detection device which detects a position of the radiation source in a revolving direction around the bed, wherein
the second tomographic image generation device uses the detected plurality of radiation source positional information in generating the second tomographic image information.

16. The computed tomography device according to claim 11, further comprising:
a rotating device for revolving the radiation source around the bed;
a rotational cycle information generation device which receives a respiratory measurement signal and a cardiac beat measurement signal of the subject, and calculates information on a rotational cycle of the radiation source based on the respiration cycle and the cardiac cycle obtained by these measurement signals; and
a rotation control device which controls rotation of the rotating device based on the rotational cycle information.

17. A computed tomography device, comprising:
a bed on which a subject is placed;
a radiation source revolving around the bed;
a plurality of radiation detectors arranged around the bed, which detect a first radiation ray caused by radiopharmaceuticals and emitted from the subject and output a first radiation detection signal, and detect a second radiation ray emitted from the radiation source and output a second radiation detection signal;
a radiation signal processing device which generates first information including first detection time information of the first radiation ray, the first positional information of the radiation detector from which the first radiation detection signal is output, and first energy information of the first radiation detection signal, from the first radiation detection signals output from the plurality of radiation detectors, and generates second information obtained from the second radiation ray, from the second radiation detection signals output from the plurality of radiation detectors;
a coincidence counting device which performs coincidence counting based on the detection time information contained in the plurality of first information, and identifies a pair of radiation detectors which output the first radiation detection signal within a setting time;
a first tomographic image generation device which, based on the each first positional information and the first detection time information of the identified pair of radiation detectors, generates first tomographic image information in a time interval defined by individually combining a plurality of first motion phases obtained by temporally dividing a respiration cycle of the subject with a plurality of second motion phases obtained by temporally dividing a cardiac cycle;
a radiation source position detection device which detects a position of the radiation source in a revolving direction around the bed;
a second tomographic image generation device which generates second tomographic image information in the plurality of time intervals based on the plurality of second information and the plurality of detected radiation source positional information;
a relative displacement information generation device which superimposes the second tomographic image information of other time interval onto the second tomographic image information of the one certain time interval among the plurality of time intervals, and calculates relative displacement information between these pieces of second tomographic image information; and
a tomographic image information superimposing device which generates first superimposed tomographic image information by superimposing the first tomographic image information of the other time interval onto the first tomographic image information of the one certain time interval by use of the relative displacement information.

18. The computed tomography device according to claim 17, further comprising:
the radiation signal processing device which generates the second information including second detection time information of the second radiation ray, second positional information on the radiation detector from which the second radiation detection signal is output, and second energy information of the second radiation detection signal, the second information being obtained from the first radiation detection signal, from the second radiation detection signal output from the plurality of radiation detectors; and
a discrimination device which receives the first information and the second information, and discriminates the first information and the second information from each other based on the first energy information and the second energy information, wherein
the coincidence counting device which performs the coincidence counting using the first detection time information of the discriminated first information, and wherein
the second tomographic image generation device which performs generation of the second tomographic image information using the discriminated second information.

19. The computed tomography device according to claim 11, wherein the radiation detector is a semiconductor radiation detector.

20. The computed tomography device according to claim 19, wherein the semiconductor radiation detector is either of cadmium tellurium (CdTe), gallium arsenide (GaAs), cadmium zinc telluride (CZT), lead iodide (PbI₂), thallium bromide (TIBr), or a combination thereof.
